(19) United States
(12) Patent Application Publication
(10) Pub. No.: US 2013/0303528 A1
(43) Pub. Date: Nov. 14, 2013

(54) CB2 Receptor Modulators in Neurodegenerative Diseases and Applications of the Same

(71) Applicant: The Board of Trustees of the University of Arkansas, Little Rock, AR (US)

(72) Inventors: Paul L. Prather, Little Rock, AR (US); John P. Crow, Little Rock, AR (US)

(21) Appl. No.: 13/853,913

(22) Filed: Mar. 29, 2013

Related U.S. Application Data
(62) Division of application No. 11/867,689, filed on Oct. 5, 2007, now abandoned.

(51) Int. Cl. A61K 31/5383; A61K 31/5377; A61K 31/4709; A61K 31/454; A61K 31/415

(52) U.S. Cl. CPC A61K 31/5383; A61K 31/454; A61K 31/415; A61K 31/4709; A61K 31/5377
USPC 514/230.2; 514/323; 514/406; 514/312; 514/235.2

(57) ABSTRACT

Compositions for the treatment of neurodegenerative diseases are disclosed. Methods of treating and monitoring progression of a neurodegenerative disease are disclosed. According to the present invention, a selective CB2 receptor modulator may be administered to a mammal for the treatment of a neurodegenerative disease.

### CROSS REFERENCES TO RELATED APPLICATIONS

This application claims the benefit of U.S. Provisional Patent Application No. 60/852,033 filed on Oct. 16, 2006, the contents of which are hereby incorporated by reference in its entirety.

### STATEMENT REGARDING FEDERALLY SPONSORED RESEARCH

Development of this invention was supported in part by National Institute on Drug Abuse grant RO1-DA13660, National Institute of Neurological Disorders and Stroke grant RO1-NS040819 and University of Arkansas for Medical Sciences Tobacco Award. The government may have certain rights in the invention contained herein.

### BACKGROUND AND BRIEF SUMMARY OF THE INVENTION

Cannabinoid receptors are members of the G-protein coupled receptor superfamily of protein receptors. There are two known cannabinoid receptor sub-types, CB1 and CB2. CB1 receptors are expressed throughout the central nervous system (CNS), while CB2 receptors are expressed predominantly in immune cells and non-neuronal tissues.

Cannabinoids are the ligands that interact with the cannabinoid receptors. Many cannabinoids produce anti-inflammatory actions via CB1 and CB2 receptors. Particular cannabinoids may be selective for either interaction with CB1 or CB2. Alternatively, particular cannabinoids may non-selectively interact with both CB1 and CB2 receptors.

There are numerous neurodegenerative diseases characterized by neuroinflammation. Examples of such neurodegenerative diseases include, but are not limited to, amyotrophic lateral sclerosis, Alzheimer's disease, Parkinson's disease, Huntington's disease and multiple sclerosis. CB2 receptors, which normally exist primarily in the periphery, are dramatically upregulated in inflamed neural tissues associated with CNS disorders.

In one embodiment of the present invention, selective CB2 receptor modulators are disclosed which are useful for the treatment of neurodegenerative diseases in a mammal. Additionally, methods of treatment of neurodegenerative diseases in a mammal are disclosed.

In one embodiment of the present invention, methods are disclosed for the identification of selective CB2 receptor modulators which are useful for the treatment of neurodegenerative diseases in a mammal.

In a further embodiment of the present invention, methods are disclosed for monitoring the progression of a neurodegenerative disease in a mammal by obtaining a biological sample from said mammal, detecting CB2 receptor expression in the biological sample and comparing the CB2 receptor expression in the biological sample to a reference sample.

### BRIEF DESCRIPTION OF THE DRAWINGS

FIG. 1. CB2, but not CB1, receptor mRNA is dramatically and selectively upregulated in the spinal cords of G93A mice, a transgenic mouse model of ALS, in a temporal pattern closely paralleling disease progression. (a) Comparison of CB2 (left panel) and CB1 (right panel) mRNA expression in spinal cords of G93A mice at various ages, relative to age-matched WT-OE, non-ALS, control mice. (inset) Separation of PCR products by 1% agarose gel electrophoresis. (b) Comparison of CB2 (left panel) and CB1 (right panel) mRNA expression in various brain regions of 120 day-old G93A mice, relative to age-matched WT-OE control mice. (c) Comparison of CB2 (left panel) and CB1 (right panel) mRNA expression in cervical and lumbar regions of spinal cords of 120 day-old G93A mice, relative to age-matched WT-OE control mice. *,**Significantly different from mRNA expression in WT-OE control mice, P<0.05, 0.01 (non-paired Student's t-test). ^{a-c}Fold-changes that are designated with different letters are significantly different, P<0.05 (analysis of variance (“ANOVA”) followed by a Dunnett's post-hoc comparison).

FIG. 2. Upregulation of CB2 receptor mRNA in spinal cords of symptomatic G93A mice is reflected by increased CB2 receptor immunoreactivity and binding. (a) Comparison of CB2 (left panel) and CB1 (right panel) protein levels by Western analysis in spinal cord membranes of 120 day-old G93A and age-matched WT-OE control mice. The relative protein levels were calculated by normalizing to actin immunoreactivity and subtracting the background intensity. (insets) Representative Western blot of CB2 (left panel) and CB1 (right panel) receptors. (b) Comparison of specific receptor binding of CB2 (left panel) and CB1 (right panel) receptors in spinal cord membranes of 120 day-old G93A and age-matched WT-OE control mice. Specific CB1 receptor binding was defined as the binding of a receptor saturating concentration of [³H]CP-55,940 (5 nM) displaced by a receptor saturating concentration of the CB1 selective ligand AM-251 (200 nM). Specific CB2 binding was defined as the binding of 5 nM [³H]CP-55,940 displaced by a receptor saturating concentration of the CB2 selective ligand AM-630 (200 nM). *,**Significantly different from the value obtained for WT-OE control mice, P<0.05, 0.01 (non-paired Student's t-test).

FIG. 3. Upregulation of CB2 receptor mRNA and protein levels in spinal cords of symptomatic G93A mice is reflected by increased function of CB2 receptors. Comparison of CB1 and CB2 receptor stimulation of [³⁵S]GTPγS binding in spinal cord membranes prepared from WT-OE (left panel) or G93A (right panel) mice. Cannabinoid-mediated G-protein activation in spinal cord membranes was measured by selective antagonism of the [³⁵S]GTPγS binding produced by a receptor saturating concentration (100 nM) of the full, non-selective CB1/CB2 agonist HU-210 (narrow hatched bars). CB1 stimulation was defined as the amount of HU-210 (100 nM) stimulated G-protein stimulation blocked by the selective CB1 antagonist O-2050 (3 μM) (filled bars). CB2 stimulation was defined as the amount of HU-210 (100 nM) stimulated G-protein stimulation blocked by the selective CB2 antagonist SR-144528 (3 μM) (open bars). CB1/CB2-stimulation was defined as the amount of HU-210 (100 nM) stimulated G-protein stimulation blocked by concurrent incubation with O-2050 (3 μM) and SR-144528 (3 μM) (wide hatched bars). *,**Significantly different from levels of [³⁵S]GTPγS binding (fmoles/mg protein) produced in response to identical conditions in WT-OE spinal cord membranes, P<0.05, 0.01 (non-paired Student's t-test). ^{a-c}Levels of [³⁵S]GTPγS binding (fmoles/mg protein) that are designated with different letters are significantly different, P<0.05 (ANOVA followed by a Dunnett's post-hoc comparison).

FIG. 4. Treatment with the selective CB2 partial agonist AM-1241, initiated at symptom onset, produces a pronounced increase in survival of G93A-SOD1 mice. (a-c) Comparison of the effects of daily treatment, initiated at symptom onset, on survival of G93A mice with (a) 5 mg/kg WIN-55,212-2 (N=6), (b) 0.3 mg/kg AM-1241 (N=14) or (c) 3.0 mg/kg AM-1241 (N=14). The response of vehicle treated control G93A mice (N=9) is represented by the open squares in each panel. (d) Comparison of the survival interval of G93A mice treated daily with vehicle or the listed drugs. *,**,***Significantly different from the vehicle treated survival curve, P<0.0249, 0.0017, 0.0005 (Kaplan-Meier survival analysis and Logrank test (Mantel-Cox). ^{a-b}Survival intervals that are designated with different letters are significantly different, P<0.05 (Kruskal-Wallis test followed by a Dunn's post-hoc comparison).

FIG. 5. Treatment with the selective CB2 partial agonist AM-1241 or selective CB2 inverse agonist AM-630, initiated at symptom onset, produces a pronounced increase in survival of G93A-SOD1 mice. (a) Comparison of the effects of daily treatment, initiated at symptom onset, on survival of G93A mice with 3.0 mg/kg AM-1241 (N=10) or 3.0 mg/kg AM-360 (N=8). The response of vehicle treated control G93A mice (N=9) is represented by the open squares in each panel. (b-c) Comparison of the survival interval of G93A mice treated daily with vehicle or the listed drugs. *,**,***Significantly different from the vehicle treated survival curve, P<0.0249, 0.0017, 0.0005 (Kaplan-Meier survival analysis and Logrank test (Mantel-Cox)). ^{a-b}Survival intervals that are designated with different letters are significantly different, P<0.05 (Kruskal-Wallis test followed by a Dunn's post-hoc comparison).

FIG. 6 demonstrates gender differences of G93A mice in survival and motor function.

FIG. 7 demonstrates the relationship of motor function to survival in AM1241 treated G93A mice.

FIG. 8 demonstrates the relationship of motor function to survival in AM630 treated G93A mice.

FIG. 9 demonstrates the relationship of motor function to survival in JTE-907 treated G93A mice.

FIG. 10 demonstrates specific GTPγS binding in end stage spinal cord homogenates from G93A-SOD1 mice.

FIG. 11 demonstrates specific GTPγS binding in end stage spinal cord homogenates from G93A-SOD1 mice.

FIG. 12 demonstrates the correlation between blockade of cannabinoid signaling and improvement of motor function and survival.

### DETAILED DESCRIPTION

Many neurodegenerative diseases are characterized by neuroinflammation. Examples of such neurodegenerative diseases include, but are not limited to, amyotrophic lateral sclerosis (ALS), Alzheimer's disease, Parkinson's disease, Huntington's disease and multiple sclerosis. The present inventors have identified pharmaceutical compositions and methods for the treatment of neurodegenerative diseases. In various embodiments of the present invention, methods of monitoring the progression of a neurodegenerative disease are disclosed. Additionally, the present inventors disclose methods of identifying molecules that are useful in the treatment of neurodegenerative diseases.

ALS is an exemplary neurodegenerative disease characterized by progressive motor neuron loss, paralysis and death within 2-5 years of diagnosis. Currently, no effective pharmacological agents exist for the treatment of this devastating disease. Neuroinflammation may accelerate the progression of ALS. Microglia are the resident macrophages of the CNS (Streit 2002). In response to CNS injury, microglia quickly convert to an “active” state during which they change to an amoeboid shape, upregulate the cell-surface expression of a variety of surface antigens and secrete several proinflammatory molecules (Hanisch 2002). As such, microglial activation in the CNS generally signifies a primary neuroinflammatory state with deleterious effects on surrounding neurons (Nelson et al. 2002). Post-mortem studies of CNS tissues obtained from ALS patients indicate that activated microglia accumulate not only in areas of profound motor neuron degeneration, but also in areas of mild damage (Ince et al. 1996).

The present inventors have utilized G93A-SOD1 mutant mice, a well-characterized animal model of ALS, to demonstrate various embodiments of the present invention. Mice which overexpress human mutant G93A-SOD1 protein develop a progressive motor neuron disease which is similar to human ALS (Gurney 1997). In the spinal cords of G93A-SOD1 (G93A) mice, an increased presence of endocannabinoids correlates with presentation of symptoms, and levels continue to escalate until the end-stage of the disease (Witting et al. 2004; Bilsland et al. 2006). Pharmacological or genetic elevation of endocannabinoid levels also slightly delays disease progression in G93A mice, while having no effect on survival (Bilsland et al. 2006). Administration of the non-selective partial cannabinoid agonists Δ⁹-Tetrahydrocannabinol (Δ⁹-THC) (Raman et al. 2004) or cannabinol (Weydt et al. 2005) are minimally successful in delaying motor impairment and prolonging survival in G93A mice after the onset of symptoms.

In particular embodiments of the present invention, the effectiveness of pharmaceutical compositions for treating and methods of treating a neurodegenerative disease were confirmed by experimental tests conducted on G93A-SOD1 mutant mice. Additionally, the present inventors demonstrate a method for monitoring progression of a neurodegenerative disease.

The present inventors demonstrate that mRNA, receptor binding and function of CB2, but not CB1, receptors are dramatically and selectively upregulated in spinal cords of G93A-SOD1 mice in a temporal pattern paralleling disease progression. Significantly, daily injections of the selective CB2 partial agonist AM-1241, initiated at symptom onset, increases the survival interval after disease onset by 56%. Additionally, mice treated with the CB2 inverse agonist AM-630 survived twice as long as those mice treated with AM-1241. Therefore, selective CB2 modulators slow motor neuron degeneration and preserve motor function and represent a novel therapeutic modality for treatment of neurodegenerative diseases. A selective CB2 modulator may be selected from the group consisting of a selective CB2 receptor partial agonist, a selective CB2 receptor antagonist and a selective CB2 receptor inverse agonist.

An agonist is a molecule that binds to a specific receptor and triggers a response in the cell expressing the receptor. An exogenous agonist mimics the action of an endogenous biochemical molecule that binds to the same receptor. A partial agonist is a molecule that binds to a specific receptor but only produces a partial physiological response compared to a full agonist.

An inverse agonist is molecule that binds to a specific receptor but exerts the opposite pharmacological effect as that of an agonist. An inverse agonist may be a partial inverse agonist or a full inverse agonist. The pharmacological effect of an inverse agonist is typically measured as the negative value of the agonist.

A receptor antagonist is a molecule that binds to a specific receptor and inhibits the function of an agonist and inverse agonist for that specific receptor. When used alone, antagonists have no intrinsic activity.

Receptor agonists, antagonists and inverse agonists may bind to the same receptor or receptor types. If an agonist has, for example, a positive effect and the inverse agonist has, for example, a negative effect, the antagonist for the receptor may take the receptor back to a neutral state by counteracting both the agonist and inverse agonist.

The data presented here provide evidence that selective CB2 modulators act as efficacious pharmacological agents with several distinct advantages for the management of neurodegenerative diseases. One benefit of potential selective CB2 modulation therapy for neurodegenerative diseases is that significant therapeutic effects are observed even when selective CB2 modulators are initiated at symptom onset. In human ALS patients, for example, drug treatment cannot begin until onset of symptoms has been established (e.g., by muscle weakness and differential diagnosis) (Strong and Rosenfeld 2003). In addition, the present inventors demonstrate that selective CB2 receptor modulators provide improved efficacy (Table 1). Due to selective CB2 receptor upregulation in the affected neural tissues (e.g., spinal cord), administration of selective CB2 modulators to mammals suffering from a neurodegenerative disease will provide enhanced therapeutic efficacy with a potential reduction in adverse effects.

One embodiment of the present invention provides a pharmaceutical composition for the treatment of a neurodegenerative disease. The neurodegenerative disease may be selected from the group consisting of amyotrophic lateral sclerosis, Alzheimer's disease, Parkinson's disease, Huntington's disease and multiple sclerosis. In a particular embodiment, the neurodegenerative disease is amyotrophic lateral sclerosis.

In one embodiment of the present invention, the pharmaceutical composition for the treatment of a neurodegenerative disease comprises a selective CB2 receptor modulator. As previously stated, the neurodegenerative disease may be selected from the group consisting of amyotrophic lateral sclerosis, Alzheimer's disease, Parkinson's disease, Huntington's disease and multiple sclerosis. In a particular embodiment, the neurodegenerative disease is amyotrophic lateral sclerosis. Additionally, the selective CB2 receptor modulator may be selected from the group consisting of a selective CB2 receptor partial agonist, a selective CB2 receptor antagonist and a selective CB2 receptor inverse agonist.

In one embodiment of the present invention, the pharmaceutical composition for the treatment of a neurodegenerative disease comprises a selective CB2 receptor partial agonist. In a particular embodiment, the selective CB2 receptor partial agonist is selected from the group consisting of AM-1241, GW-405833 and JWH-015.

In one embodiment of the present invention, the pharmaceutical composition for the treatment of a neurodegenerative disease comprises a selective CB2 receptor antagonist. In a particular embodiment, the selective CB2 receptor antagonist is WIN-55,212-3.

In one embodiment of the present invention, the pharmaceutical composition for the treatment of a neurodegenerative disease comprises a selective CB2 receptor inverse agonist. In a particular embodiment, the selective CB2 receptor inverse agonist is selected from the group consisting of AM-630, SR-144528 and JTE-907.

In cases where the pharmaceutical composition is sufficiently basic or acidic to form stable, nontoxic acid or base salts, administration of the pharmaceutical compositions as salts may be appropriate. Examples of pharmaceutically acceptable salts include organic acid addition salts formed with acids that form a physiologically acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

The pharmaceutical compositions may be administered to a mammal, for example, a human, in a variety of forms. Administration may be, for example oral, parenteral, intravenous, intramuscular, topical or subcutaneous.

In a further embodiment of the present invention, the inventors disclose a method of treating a neurodegenerative disease in a mammal comprising administering a selective CB2 receptor modulator to a patient in need thereof. The neurodegenerative disease may be selected from the group consisting of amyotrophic lateral sclerosis, Alzheimer's disease, Parkinson's disease, Huntington's disease and multiple sclerosis. In one particular embodiment, the neurodegenerative disease is amyotrophic lateral sclerosis.

In various embodiments of the method of treating a neurodegenerative disease in a mammal, the selective CB2 receptor modulator is selected from the group consisting of a selective CB2 receptor partial agonist, a selective CB2 receptor antagonist and a selective CB2 receptor inverse agonist.

In various embodiments of the method of treating a neurodegenerative disease in a mammal, the selective CB2 receptor modulator may be administered to the mammal in a dose of about 0.3 mg/kg, or about 1.0 mg/kg, or about 1.5 mg/kg, or about 2.0 mg/kg/day, or about 2.5 mg/kg/day, or about 3.0 mg/kg/day, or about 3.5 mg/kg/day, or about 4.0 mg/kg/day, or about 4.5 mg/kg/day, or about 5.0 mg/kg/day.

In one embodiment of the method of treating a neurodegenerative disease in a mammal, the selective CB2 receptor modulator is a selective CB2 receptor partial agonist. In a particular embodiment, the selective CB2 receptor partial agonist is selected from the group consisting of AM-1241, GW-405833 and JWH-015.

In one embodiment of the method of treating a neurodegenerative disease in a mammal, the selective CB2 receptor modulator is a selective CB2 receptor antagonist. In a particular embodiment, the selective CB2 receptor antagonist is WIN-55,212-3.

In one embodiment of the method of treating a neurodegenerative disease in a mammal, the selective CB2 receptor modulator is a selective CB2 receptor inverse agonist. In a particular embodiment, the selective CB2 receptor inverse agonist is selected from the group consisting of AM-630, SR-144528 and JTE-907.

In yet a further embodiment of the present invention, the inventors disclose a method of monitoring progression of a neurodegenerative disease comprising obtaining a biological sample from a mammal, detecting CB2 receptor protein in said biological sample and comparing the expression level of the CB2 receptor protein in the biological sample to a reference sample. Detecting CB2 receptor protein may be accomplished through numerous techniques known to those of skill in the art, including, but not limited to, ELISA, western blot, immuno-dot blot, immunohistochemistry, immunoaffinity and ligand affinity assays.

In yet a further embodiment of the present invention, the inventors disclose a method of monitoring progression of a neurodegenerative disease comprising obtaining a biological sample from a mammal, detecting CB2 receptor mRNA in said biological sample and comparing the expression level of the CB2 receptor mRNA in the biological sample to a reference sample. Detecting CB2 receptor mRNA may be accomplished through numerous techniques known to those of skill in the art, including, but not limited to, Northern blot, dot blot, RT-PCR, and in situ hybridization.

In various embodiments of the present invention, a biological sample may be selected from the group consisting of tissue biopsy, saliva, cerebrospinal fluid, tears, blood, and urine.

In yet a further embodiment of the present invention, the inventors disclose a method of identifying molecules for the treatment of a neurodegenerative disease comprising identifying selective CB2 receptor modulators. The method may be useful to identify molecules for the treatment of a neurodegenerative disease selected from the group consisting of amyotrophic lateral sclerosis, Alzheimer's disease, Parkinson's disease, Huntington's disease and multiple sclerosis. In a particular embodiment, the neurodegenerative disease may be amyotrophic lateral sclerosis.

In one embodiment of the method of identifying molecules for the treatment of a neurodegenerative disease the molecule is a selective CB2 receptor modulator selected from the group consisting of a selective CB2 receptor partial agonist, a selective CB2 receptor antagonist and a selective CB2 receptor inverse agonist.

### EXAMPLES

The following examples are further illustrative of the present invention, but it is understood that the invention is not limited thereto. The non-selective CB1/CB2 agonists examined in this study were CP-55,940, (−)-cis-3-[2-Hydroxy-4-(1,1-dimethylheptyl)-phenyl]-trans-4-(3-hydroxypropyl)-cyclohexanol), WIN-55,212-2, [2,3-Dihydro-5-methyl-3-[(morpholinyl)methyl]pyrrolo-[1,2,3-de]-1,4-benzoxazin-yl]-(1-naphthalenyl)methanone, HU-210, (−)-11-Hydroxy-delta(8)-tetrahydrocannabinol-dimethylheptyl, and 2-arachidonoyl glycerol (2-AG) [(5Z,8Z,11Z,14Z)-5,8,11,14-Eicosatetraenoic acid, 2-hydroxy-1-(hydroxymethyl)ethyl ester].

The selective CB1 agonist employed was ACEA, (all Z)—N-(2-cycloethyl)-5,8,11,14-eicosatetraenamide.

The selective CB1 antagonists used were AM-251 (N-(Piperidin-1-yl)-5-(4-iodophenyl)-1-(2,4-dichlorophenyl)-4-methyl-1H-pyrazole-3-carboxamide and O-2050 ((6aR,10aR)-3-(1-Methanesulfonylamino-4-hexyn-6-yl)-6a,7,10,10a-tetrahydro-6,6,9-trimethyl-6H-dibenzo[b,d]pyran).

The selective CB2 partial agonists examined were AM-1241 ((R,S)-(+)-(2-Iodo-5-nitrobenzoyl)-[1-(1-methyl-piperidin-2-ylmethyl)-1H-indole-3-yl]methanone) and GW-405833, (2,3-dichloro-phenyl)-[5-methoxy-2-methyl-3-(2-morpholin-4-yl-ethyl)-indol-1-yl]-methanone.

The selective CB2 antagonists/inverse agonists used were AM-630, 6-iodo-2-methyl-1-[2-(4-morpholinyl)ethyl]-1H-indol-3-yl](4-methoxyphenyl)methanone and SR-144528, N-[(1S)-endo-1,3,3-Trimethylbicyclo[2.2.1]heptan-2-yl]-5-(4-chloro-3-methylphenyl)-1-(4-methoxybenzyl)-pyrazole-3-carboxamide.

All drugs were obtained from Tocris Bioscience except HU-210 and SR-144528, both of which were provided from the National Institute on Drug Abuse (NIDA) drug inventory supply and control system.

Additional pharmaceutical compounds that are useful in pharmaceutical compositions and methods of the present invention include, but are not limited to, WIN-55,212-3, which is the (S)(−) isomer of WIN-55,212-2, [2,3-Dihydro-5-methyl-3-[(morpholinyl)methyl]pyrrolo-[1,2,3-de]-1,4-benzoxazin-yl]-(1-naphthalenyl)methanone, JWH-015, (2-Methyl-1-propyl-1H-indol-3-yl)-1-naphthalenylmethanone, and JTE-907 (Iwamura, 2001).

Hemizygous transgenic male mice with the G93A mutation of the human SOD1 gene (G1H/1) mutation (B6SJL-TgN (SOD1-G93A) 1 Gur) were obtained from Jackson Laboratories and were bred locally with female B6SJL mice (Jackson Laboratory), according to the protocol obtained from the vendor. To decrease the inherent variability in disease onset and survival with these mice, littermate transgenic males (rather than random males from several different litters) were selected to sire subsequent generations of mice. Within three generations, the variability was all but eliminated such that the transgenic mice develop characteristic hind limb weakness at 90 days of age (+/−1-2 days) and progress to end-stage disease (requiring sacrifice) within 18-30 days after onset; this has remained relatively constant for the last eight generations of mice.

Determination of symptom onset, randomization and drug treatment of G93A mice. Symptom onset was assessed by blinded observation of changes in hind limb gait; these changes are related to the mouse's inability to support its weight on its hind limbs. At onset, mice initially place their weight on the toes and then quickly fall to full foot placement (as opposed to healthy mice which walk and run on the hind toes); this “toe-to-heel” walking pattern produces an asymmetric gait between hind and fore limbs and a characteristic “wobbling” gait. Mouse groups were randomized based on symptom onset and alternately placed in control and treated groups, e.g., the first mouse to develop hind limb weakness was placed in the control group, the second was injected with test compound and placed in the treatment group, and so on. The net effect of this type of randomization was to create groups with mean onset ages which are virtually identical, thereby allowing the use smaller numbers of mice (typically 10-13 per group) and still maintain sufficient statistical power. By definition, the onset administration paradigm employed was focused on what we term the “survival interval”—namely the time from onset to end-stage sacrifice. Because both drug- and vehicle-treated groups were derived from the same groups of age-matched mice, survival results were properly normalized by comparing survival intervals of drug-treated to survival intervals of vehicle-treated groups to determine survival interval ratios.

All drugs and vehicle were administered once daily by the i.p. route beginning the first day of symptom onset. AM-1241, AM630, JTE-907 and WIN-55,212-2 have very poor water solubility and require a vehicle which is both capable of dissolving the drug and is biocompatible (with chronic dosing). Other groups have used complex vehicles composed of polyethoxylated vegetable oils and/or ethanol/glycerol/water mixtures. We tested a number of traditional vehicles such as ethanol/water, glycerol, polyethylene glycol, and high purity olive oil. Stable dissolution of AM-1241, AM630, JTE-907 and WIN-55,212-2 was achieved only with olive oil, thus it was selected as the vehicle for these studies. Two different concentrations of AM-1241 (1 mg/ml and 0.1 mg/ml) and one WIN-55,212-2 concentration (1 mg/ml) were prepared in order to minimize the volume of olive oil that was injected IP.

Mice were sacrificed when any of the following criteria were met: (1) inability to right themselves within 30 seconds when placed on their sides; (2) inability to eat or drink, or move toward food and water placed in low-rimmed dishes on cage floor; (3) loss of more than 10% of total body weight in 24 hours; (4) gross loss of grooming behavior; or (5) labored breathing. Criteria for death were confirmed by a second investigator who is blinded to the group identity of each mouse. The age of symptom onset was subtracted from the age at death for each mouse, and a mean survival interval was calculated for each group. By calculating the ratio of the survival interval of treated groups to the survival interval of untreated littermate controls, a X-fold increase in survival was readily determined.

Brain regions were dissected from fresh mouse brains placed on an ice-cooled surface. Spinal cords, individual brain regions or spleen were suspended in a homogenization buffer containing 50 mM Hepes, pH 7.4, 3 mM MgCl₂, and 1 mM EGTA. Using a 7 mL Dounce glass homogenizer (Wheaton), samples were subjected to 10 complete strokes and centrifuged at 18,000 rpm for 10 min at 4° C. After repeating the homogenization procedure twice more, the samples were resuspended in Hepes buffer (50 mM, pH 7.4) and subjected to 10 strokes utilizing a 7 mL glass homogenizer. Membranes were stored in aliquots of approximately 1 mg/mL at −80° C.

Beginning on the first day of observing symptoms, mice were tested for motor function biweekly until sacrifice. Motor function testing was initiated by gently placing animals on a horizontal wire mesh screen. The screen was slowly rotated to be perpendicular to the ground (90°). The duration that mice were able to hang onto the perpendicular wire mesh up to a maximum of 120 seconds was recorded. Because vehicle treated mice on average lost the ability to cling to the wire mesh for 60 seconds in the middle of their survival curve, this “all or none” response was utilized to compare motor function across all treatment groups. Kaplan-Meier time-to-event statistics were employed to analyze the data. The event was defined as the number of days after symptom onset that each individual animal was no longer able to cling to the wire mesh screen for 60 seconds.

Quantitative real time PCR (qRT-PCR) was performed as follows: Total RNA was isolated from G93A and WT-OE mice tissues using an RNeasy minikit and QiaShredder columns (Qiagen Operon). Genomic DNA contamination was eliminated using DNAse-free (Ambion). Total RNA (1 μg) was reverse transcribed according to commercial instructions (iScript cDNA synthesis kit; Biorad) to generate cDNA at 25° C. for 5 min, followed by 42° C. for 30 min and 85° C. for 5 min. The cDNA sequences for the appropriate targets were amplified using the polymerase chain reaction and corresponding primers. The PCR primers for CB1 were CB1 Forward 5′-CTGAACTCCACCGTGAACC-3′ and CB1 Reverse 5′-TTATTGGCGTGCTTGTGC-3′, which produced a 152 base pair PCR product. The PCR primers for CB2 were CB2 Forward 5′-TGCACTGGCTCTCATGG-3′ and CB2 Reverse 5′-GAGCGAATCTCTCCACTCC-3′, which produced a 144 base pair PCR product. The control PCR primers for GAPDH were GAPDH Forward 5′-GGGAAGCTCACTGGCATGG-3′ and GAPDH Reverse 5′-CTTCTTGATGTCATCATACTTGGCAG-3′, which produced a 123 base pair PCR product.

The PCR mixture contained 1×iQ SYBR Green Supermix (Biorad), 200 nmol/L each of forward and reverse primers, and 10 ng of template. After initial denaturation at 95° C. for 3 min, the following temperature-cycling profile for the amplification was used (40 cycles): 95° C. for 10 sec denaturing and 62° C. for 1 min for annealing and extension. Melting curve analysis was accomplished in 80 cycles. The steps included 95° C. for 1 min for denaturation, 55° C. for 1 min to permit final extension, and 0.5° C. temperature increments for 10 sec each cycle from 55 to 95° C. Amplified cDNA products were analyzed using iCycler software (Biorad).

Western blots. To identify CB1 and CB2 receptors, each sample containing 100 μg of spinal cord membrane protein was separated by SDS-PAGE on 10% (w/v) polyacrylamide mini-gels. Prior to separation, samples were re-suspended in 40 μL of electrophoresis loading buffer (0.065 M Tris HCl, pH 6.8, 2% SDS, 10% glycerol, 5% 2-mercaptoethanol), and heated at 90° C. for 2 min. The ECL method of immunoblotting was employed (GE Healthcare/Amersham Biosciences). Gels were transferred to Hybond-ECL nitrocellulose membranes and incubated overnight at 4° C. with 10% milk in blotting buffer (TBS-0.1%) (Tris HCl, pH 7.6, 25 mM; NaCl, 0.09%; Tween-20, 0.1%). Blots were then washed 3 times (5 min each) with TBS-0.1% and incubated with primary antibodies (1:200) overnight at 4° C. while shaking. For selected blots, the appropriate blocking peptide (1:100) was incubated with the respective primary antibody for 1 hr at room temperature prior to incubation with blots. The primary antibody solutions were removed and blots washed as described previously. Secondary antibody (donkey anti-rabbit horseradish peroxidase, 1:5000) was added and incubated for 4 hrs, with shaking. The secondary antibody was removed and blots washed as described. Blots were incubated for 1 min with equal volumes of ECL detection reagents 1 and 2. Chemiluminescence was captured for 2 hrs and saved as a TIFF file by a Fluorochem 8900 MultiImage Light Cabinet (Alpha Innotech Corp.). The captured images were digitized and the relative cannabinoid receptor levels compared after densitometry analysis (Scion NIH Image 1.63). The relative protein levels were calculated by normalizing to actin immunoreactivity and subtracting the background intensity.

The primary antibodies and blocking peptides for both the CB1 and CB2 receptor were purchased from Cayman Chemical. The CB1 receptor polyclonal antibody (Catalog No. 10006590) was raised against the C-terminal amino acids 461-472 of the human CB1 receptor. This antigen is identical to the corresponding sequence in murine, rat, canine and bovine species. The CB2 receptor polyclonal antibody (Catalog No. 101550) was raised against amino acids 20-33 in a sequence between the N-terminus and the first transmembrane domain of the protein of the human CB2 receptor. Human and murine CB2 receptors exhibit 82% homology at the amino acid level over the complete protein. CB1 (Catalog No. 10006591) and CB2 (Catalog No. 301550) blocking peptides were derived from the CB1 and CB2 receptor sequences used as antigens for production of the respective polyclonal antiserum.

Cannabinoid receptor binding assays were performed as follows: Each binding assay contained 30 μg of spinal cord membrane protein in a final volume of 1 mL in binding buffer (50 mM Tris, 0.1% bovine serum albumin, 5 mM of MgCl₂, pH 7.4), as described previously (Prather et al. 2000b). [³H]CP-55,940 (180 Ci/mmol, Perkin Elmer Life and Analytical Sciences) binds with equivalent affinity to CB1 and CB2 receptors with an approximate Kᵢ of 0.5 nM (Shoemaker et al. 2005). Specific CB1 receptor binding was defined as the binding of a receptor saturating concentration of [³H]CP-55,940 (5 nM) displaced by a receptor saturating concentration of the CB1 selective ligand AM-251 (200 nM). AM-251 displays high affinity for CB1 receptors with a Kᵢ value of about 7 nM, whereas its affinity at CB2 receptors is over 300-fold weaker (Gatley et al. 1997). Specific CB2 binding was defined as the binding of 5 nM [³H]CP-55,940 displaced by a receptor saturating concentration of the CB2 selective ligand AM-630 (200 nM). AM-630 binds CB2 receptors with high affinity [Kᵢ value of about 20 nM, (Shoemaker et al. 2005)], whereas its affinity for CB1 receptors is more than 165-fold less (Hosohata et al. 1997). All binding experiments were performed in triplicate. Reactions were terminated by rapid vacuum filtration through Whatman GF/B glass fiber filters followed by two washes with ice-cold binding buffer. 4 mL of Scintiverse® (Fisher Scientific) was added to the filters and radioactivity quantified by scintillation counting.

[³⁵S]GTPγS binding assays were performed as described previously (Prather et al. 2000a) in a buffer containing 20 mM Hepes, 100 mM NaCl, and 10 mM MgCl₂ at pH 7.4. Each binding reaction contained 10 μg of spinal cord membrane protein, the presence or absence of cannabinoid ligands (see following), plus 0.1 nM [³⁵S]GTPγS (1250 Ci/mmol, Perkin Elmer Life and Analytical Sciences) and 10 μM of GDP to suppress basal G-protein activation. Reactions were incubated for 2 hrs at 30° C. Non-specific binding was defined by binding observed in the presence of 10 μM of non-radioactive GTPγS. The reaction was terminated by rapid vacuum filtration through glass fiber filters followed by two washes with ice-cold assay buffer. 4 mL of Scintiverse® (Fisher Scientific) was added to the filters and radioactivity quantified by scintillation counting.

Cannabinoid-mediated G-protein activation in spinal cord membranes was measured by selective antagonism of the [³⁵S]GTPγS binding produced by a receptor saturating concentration (100 nM) of the full, non-selective CB1/CB2 agonist HU-210. HU-210 binds with equivalent affinity to CB1 and CB2 receptors with an approximate Kᵢ of 0.5 nM (Felder et al. 1995; Breivogel et al. 2001). In these studies, we first determined the minimal concentration of the neutral CB1 antagonist O-2050 (Gardner and Mallet 2006) required to completely block CB1-mediated G-protein activation by HU-210. This was accomplished by antagonism experiments employing membranes prepared from mouse cortex as a relatively pure source of CB 1 receptors. In these studies, it was determined that 3 μM of O-2050 was the minimal concentration required to completely block HU-210-mediated (100 nM) G-protein activation by CB1 receptors in cortical membranes (data not shown). Next, the minimal concentration of the selective CB2 antagonist SR-144528 (Rinaldi-Carmona et al. 1998) required to completely block CB2-mediated G-protein activation by HU-210 was determined. This was accomplished by antagonism experiments employing membranes prepared from CHO-CB2 cells (Shoemaker et al. 2005) as a pure source of CB2 receptors. In these studies, it was shown that 3 μM of SR-144528 was the minimal concentration required to completely block HU-210-mediated (100 nM) G-protein activation by CB2 receptors in CHO-CB2 membranes (data not shown). Therefore, employing spinal cord membranes harvested from WT-OE and G93A mice, CB1-selective stimulation was defined as the amount of O-2050 (3 μM) sensitive G-protein stimulation produced by HU-210 (100 nM). CB2-selective activation was defined as the amount of SR-144528 (3 μM) sensitive G-protein stimulation produced HU-210 (100 nM).

The selective antagonism method described here was developed in response to many failed attempts to demonstrate consistent, measurable G-protein activation with the selective CB1 agonist ACEA (Hillard et al. 1999) or the CB2 partial agonists GW-405833 (Valenzano et al. 2005) and AM-1241 (Yao et al. 2006) in mouse spinal cord membranes (data not shown). While these observations were surprising for the full CB1 agonist ACEA (Hillard et al. 1999), both GW-405833 and AM-1241 have been reported to act as partial agonists in several in vitro assays (Valenzano et al. 2005; Yao et al. 2006). In any case, it is likely that the poor G-protein stimulation produced by partial agonists in the present study is due to less than optimal experimental conditions and/or a relatively low density of cannabinoid receptors expressed in spinal cord membranes, leading to reduced receptor-mediated responses.

Statistical analysis. All curve-fitting and statistical analysis was conducted by employing the computer program GraphPad Prism® v4.0b (GraphPad Software, Inc.; San Diego, Calif.). All data are expressed as mean±S.E.M. To compare three or more groups of data that follow a Gaussian distribution, statistical significance of the data was determined by a one-way ANOVA, followed by post-hoc comparisons using a Dunnett's test. To compare two groups of data that follow a Gaussian distribution, the non-paired Student's t-test was utilized. To compare three or more groups of data that do not follow a Gaussian distribution, statistical significance of the data was determined by the non-parametric Kruskal-Wallis test, followed by post-hoc comparisons using a Dunnett's test. Kaplan-Meier survival analysis and the Logrank (Mantel-Cox) test were used for survival comparisons.

### Example 1

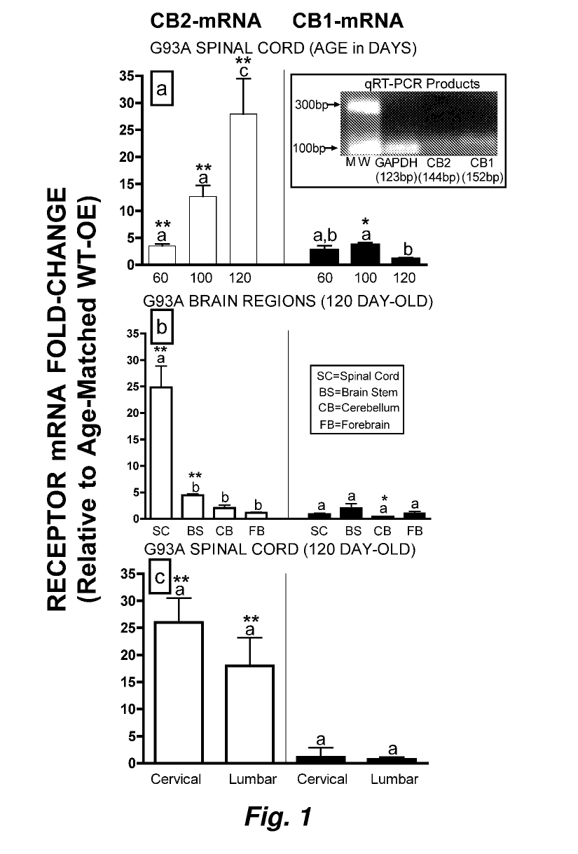

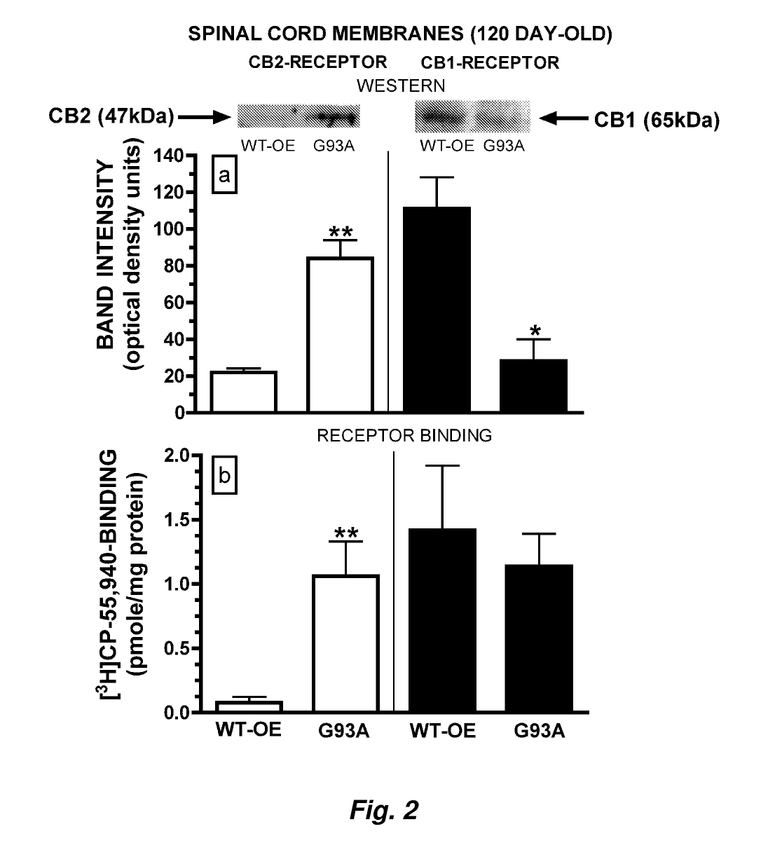

Initial experiments examined the spatial and temporal expression of CB2 receptors in the CNS of G93A mice (FIGS. 1 and 2). First, qRT-PCR compared CB1 and CB2 receptor mRNA expression in the spinal cords of G93A mice relative to age-matched mice overexpressing the human Wild-Type-SOD1 gene (WT-OE) (FIG. 1A). The amplification efficiency of the primers designed for the targets (CB1, CB2) and reference (GAPDH) cDNAs was equivalent (data not shown) and the PCR products were of the predicted size (FIG. 1A, inset). Therefore, the comparative Ct method was employed for mRNA comparison (Giulietti et al. 2001). The expression level of CB1 mRNA (right panel) is slightly elevated in the spinal cords of 100 (3.7±0.4-fold, P<0.05, N=6), but not 60 (2.7±0.8-fold, N=3) or 120 (1.1±0.2-fold, N=3) day-old G93A mice, compared to age-matched WT-OE control animals. In addition, a small but significant (P<0.05) decrease of CB1 mRNA occurs in end-stage G93A mice (120 days of age), relative to 100 day-old G93A mice. In contrast, CB2 mRNA (left panel) is significantly elevated in the spinal cords of 60 (3.6±0.3-fold, P<0.01, N=3), 100 (12.6±2.0-fold, P<0.01, N=6) and 120 (27.9±6.5-fold, P<0.01, N=3) day-old G93A mice relative to age-matched WT-OE controls. Furthermore, the elevation in CB2 mRNA is age-dependent, increasing slightly in 60 day-old mice prior to symptom onset and rising to the highest levels in 120 day-old mice (P<0.01).

To determine if CB2 mRNA upregulation in the CNS of G93A mice is correlated in any way to disease pathology, cannabinoid receptor mRNA expression was examined in the spinal cord (SC), brain stem (BS), cerebellum (CB) and forebrain (FB) of end-stage (120 day-old) G93A mice, relative to age-matched WT-OE controls (FIG. 1B). While CB1 mRNA (right panel) is slightly decreased in the cerebellum of end-stage G93A mice relative to WT-OE controls (0.4±0.1-fold, P<0.05, N=3), this reduction is not significantly different when compared to CB1 mRNA changes in all other brain regions of G93A mice (SC: 0.8±0.3-fold, N=6; BS: 2.0±0.9-fold, N=3; FB: 1.0±0.4-fold, N=4). In sharp contrast, CB2 mRNA is significantly increased only in the spinal cord (24.8±4.1-fold, P<0.01, N=5) and brainstem (4.5±0.3-fold, P<0.01, N=3), but not in cerebellum (2.0±0.6-fold, N=3) or forebrain (1.1±0.4-fold, N=4). CB2 mRNA upregulation is much greater in the spinal cord than in the brainstem (P<0.01) of G93A mice, consistent with disease pathogenesis.

Cannabinoid receptor mRNA expression in lumbar and cervical regions of spinal cords of end-stage G93A mice was next examined (FIG. 1C). CB1 mRNA levels (right panel) are unchanged in either the cervical (1.1±1.8-fold, N=3) or lumbar (0.72±0.4-fold, N=3) spinal cord regions. Unlike the reported regional distribution of endocannabinoids (Witting et al. 2004), CB2 receptor mRNA upregulation is similar in both the cervical (25.6±4.5-fold, N=3) and lumbar (18.0±5.2-fold, N=3) regions of G93A spinal cords when compared to age-matched WT-OE control mice.

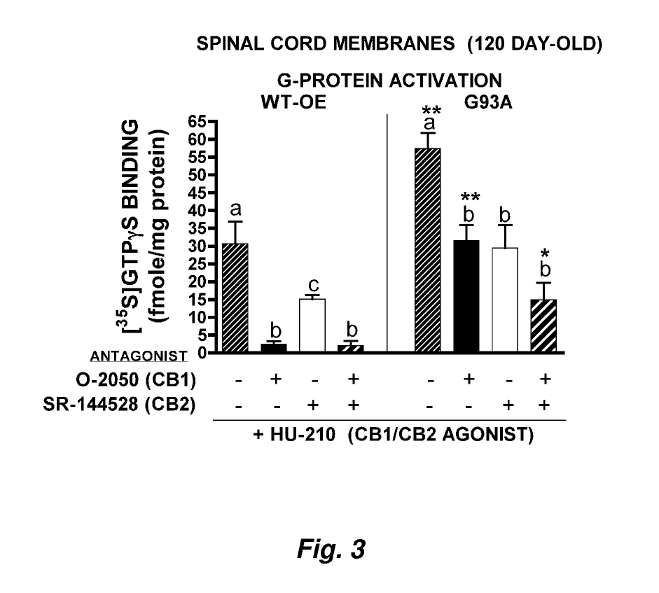

The density and function of cannabinoid receptors was next examined in membranes prepared from spinal cords using Western analysis (FIG. 2A), receptor binding (FIG. 2B) and [³⁵S]GTPγS binding (FIG. 3) assays. In initial optimization studies, the CB1 receptor antibody identified an immunoreactive band in membranes prepared from mouse cortex (a relatively pure source of CB1 receptors), but not from CHO-CB2 membranes, with a molecular weight predicted for CB1 receptors of approximately 65 kDa (data not shown). In contrast, a 47 kDa immunoreactive band corresponding to the predicted molecular weight for CB2 receptors was recognized by the CB2 receptor antibody in membranes prepared from CHO-CB2 cells (a pure source of CB2 receptors), but not from mouse cortex (data not shown). In spinal cord membranes prepared from WT-OE and G93A mice (FIG. 2A), selective antibodies identified immunoreactive bands with the predicted molecular weight for CB2 (left panel inset) or CB1 (right panel inset) receptors. Furthermore, the band recognized by both antibodies was eliminated upon preincubation of antibodies with an excess of the appropriate blocking peptide (data not shown). Although little CB2 receptor immunoreactivity is present in spinal cords of 120 day-old WT-OE mice (22.0±2.1 OD units, N=3), approximately 4-fold greater CB2 receptor density (P<0.01) is observed in end-stage G93A animals (84.0±9.9 OD units, N=3). In contrast, CB1 receptor immunoreactivity is decreased (P<0.05) almost 4-fold in spinal cord membranes of 120 day-old G93A (28.0±12.0 OD units, N=3), relative to WT-OE (111.0±17.0 OD units, N=3) control mice.

Cannabinoid receptor binding experiments (FIG. 2B) were conducted to confirm the results observed from Western analysis. Similar to results reported for mRNA and Western analysis, predominantly CB1 (1.42±0.5 pmole/mg, N=3) and much less CB2 (0.077±0.046 pmole/mg, N=3) receptors are present in spinal cord membranes of 120 day-old WT-OE control mice. In agreement with elevated CB2 mRNA and immunoreactivity, CB2 receptor density also is increased over 13-fold in the spinal cords of 120 day-old G93A mice (1.06±0.27 pmole/mg, P<0.01, N=3), relative to that observed in age-matched WT-OE controls. Similar to decreased immunoreactivity, CB1 receptor density also is reduced slightly, although not significantly, by 20% (to 1.14±0.25 pmole/mg, N=3) in 120 day-old G93A relative to age matched WT-OE control mice.

To determine if the upregulated CB2 receptors in G93A spinal cord membranes are functional, G-protein activation assays were conducted (FIG. 3). We initially attempted to compare CB1 and CB2 receptor activation of G-proteins between WT-OE and G93A spinal cord membranes by conducting [³⁵S]GTPγS binding assays in the presence of selective agonists. However, after considerable effort, we were unable to demonstrate consistent, measurable G-protein activation with the selective CB1 agonist ACEA (Hillard et al. 1999) or the CB2 partial agonists GW-405833 (Valenzano et al. 2005) and AM-1241 (Yao et al. 2006) in mouse spinal cord membranes (data not shown). Therefore, G-protein activation produced by CB1 and CB2 receptors was instead quantified by selectively antagonizing the [³⁵S]GTPγS binding produced by the CB1/CB2 full agonist HU-210 (Felder et al. 1995; Breivogel et al. 2001) with the CB1 antagonist 0-2050 (Gardner and Mallet 2006) or the CB2 antagonist SR-144528 (Rinaldi-Carmona et al. 1998).

### Example 2

In WT-OE spinal cord membranes (FIG. 3, left panel), stimulation of CB1/CB2 receptors by HU-210 produces 30.7±6.2 fmole/mg protein (N=4) of [³⁵S]GTPγS binding to G-proteins. Co-incubation with the CB1 selective antagonist O-2050 almost completely blocks G-protein stimulation by HU-210 (to 2.5±0.8 fmole/mg protein, P<0.01, N=4). Interestingly, the CB2 selective antagonist SR-144528 also significantly reduces HU-210 stimulation by approximately 50% (to 15.1±1.1 fmole/mg protein, P<0.05, N=4). As might have been anticipated, co-incubation of HU-210 with both antagonists concurrently also reduces G-protein activation by over 90% (to 2.1±1.3 fmole/mg protein, P<0.01, N=4). Collectively, these data indicate that the stimulation G-proteins produced by HU-210 in WT-OE spinal cord membranes occurs primarily via activation of CB1 receptors. Although the partial reduction of G-protein stimulation by HU-210 in the presence of the CB2 selective antagonist SR-144528 suggests that CB2 receptors may also participate, it is possible that the observed results might be due to non-selective blockade of CB1 receptors by the 3 μM concentration of SR-144528 employed in the assay.

In G93A spinal cord membranes (FIG. 3, right panel), stimulation of CB1/CB2 receptors by HU-210 produces a significantly greater increase in [³⁵S]GTPγS binding to G-proteins (57.4±4.4 fmole/mg protein, P<0.01, N=4) relative to that observed in WT-OE membranes. Furthermore, in G93A membranes, co-incubation of HU-210 with the CB1 selective antagonist O-2050 reduces G-protein stimulation by only 46% (to 31.5±4.4 fmole/mg protein, P<0.05, N=4), compared to near complete blockade in WT-OE membranes. Importantly, although the % blockade of HU-210-induced G-protein activation by O-2050 in G93A membranes is half of that observed in WT-OE membranes (45 versus 92%), the net reduction in fmoles of activated G-proteins by O-2050 is almost identical between membrane preparations. In other words, O-2050 reduced HU-210-induced G-protein activation by 28.3 fmoles/mg protein in WT-OE membranes (30.7−2.5=28.3 fmole/mg protein) and 25.9 fmole/mg protein in G93A membranes (57.4−31.5=25.9 fmole/mg protein). This indicates that CB1 receptors activate similar levels of G-proteins in both WT-OE and G93A tissues. The CB2 selective antagonist SR-144528 also significantly (P<0.05, N=4) reduces HU-210 G-protein stimulation in G93A membranes by 49%, to 29.5±6.4 fmole/mg protein. In contrast to that observed for CB1 receptors, the net reduction in fmoles of activated G-proteins by SR-144528 is markedly different between membrane preparations. For example, SR-144528 reduces G-protein activation by 15.6 fmoles/mg protein in WT-OE membranes (30.7−15.1=15.6 fmole/mg protein) and 27.9 fmole/mg protein in G93A membranes (57.4−29.5=27.9 fmole/mg protein). This suggests that CB2 receptors activate approximately twice the amount of G-proteins in G93A, relative to WT-OE spinal cord membranes. Very interestingly, although co-incubation of HU-210 with both antagonists concurrently reduces G-protein activation to a level lower than that obtained with either antagonist alone, a significant level of HU-210-activated G-proteins can not be blocked under these conditions (14.9±4.8 fmole/mg protein, N=4). These data indicate that HU-210 may activate G-proteins via a non-CB1/CB2 receptor in spinal cord membranes prepared from G93A, but not WT-OE mice.

### Example 3

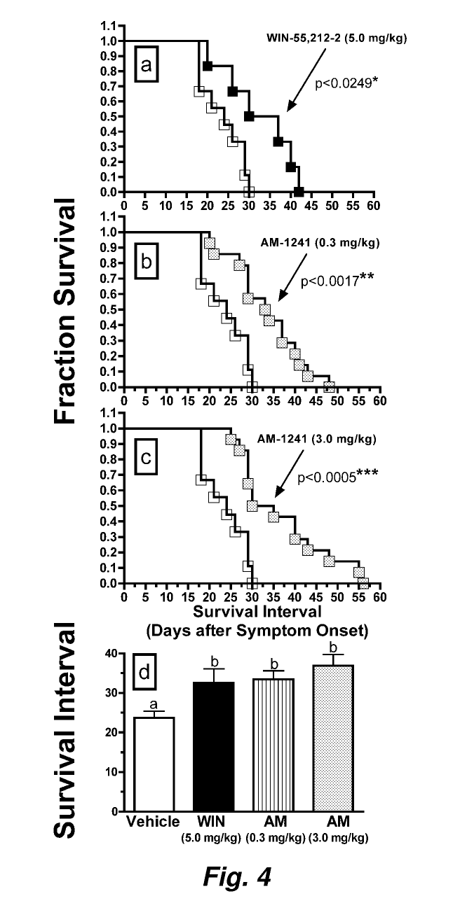

The effect of chronic administration of cannabinoids on the survival of G93A mice was next examined (FIG. 4). Two cannabinoid agonists were tested, WIN-55,212-2 and the partial agonist AM-1241. WIN-55,212-2 exhibits a slightly higher affinity for human CB2 (3.3 nM), when compared to CB1 (62.3 nM) receptors (Felder et al. 1995). In contrast, AM-1241 displays over an 80-fold higher affinity for CB2 (7 nM), relative to CB1 (580 nM) receptors (Yao et al. 2006). Mice were administered daily i.p. injections, beginning at onset of symptoms, with one of four treatments; vehicle (FIG. 4A-C, n=9), the relatively non-selective CB1/CB2 agonist WIN-55,212-2 (5 mg/kg, FIG. 4A, N=6), the selective CB2 partial agonist AM-1241 (0.3 mg/kg, FIG. 4B, N=14) or AM-1241 (3 mg/kg, FIG. 4C, N=14). The number of days between symptom onset and animal sacrifice was measured (e.g., survival interval). In humans, this is analogous to the time between diagnosis of ALS and death, ranging from 2-5 years. Mice injected with vehicle (open squares in all panels) survive from 18-30 days following symptom onset, with an average survival interval of 23.7+/−1.7 days (FIG. 4D). Treatment at onset with the non-selective CB1/CB2 agonist WIN-55,212-2 produces a significant rightward-shift in the survival curve (P<0.0249), reflected by an increase of 8.8 days in the survival interval (32.5+/−3.6 days, P<0.0496) (FIG. 4A). Onset administration with either 0.3 (FIG. 4B, P<0.0017) or 3.0 mg/kg (FIG. 4C, P<0.0005) of the selective CB2 partial agonist AM-1241 results in a highly significant extension of survival. Mice receiving daily injections of 0.3 and 3 mg/kg AM-1241 live an average of 9.7 (33.4+/−2.2 days, P<0.0081) and 13.2 (36.9+/−2.8 days, P<0.0022) days longer after symptom onset than vehicle treated controls, respectively (FIG. 4D).

### Example 4

Treatment with the selective CB2 partial agonist AM-1241 or selective CB2 inverse agonist AM-630, initiated at symptom onset, produces a pronounced increase in survival of G93A-SOD1 mice. (a) Comparison of the effects of daily treatment, initiated at symptom onset, on survival of G93A mice with 3.0 mg/kg AM-1241 (N=10) or 3.0 mg/kg AM-630 (N=8). The response of vehicle treated control G93A mice (N=9) is represented by the open squares in each panel. (b-c) Comparison of the survival interval of G93A mice treated daily with vehicle or the listed drugs.

When compared to the efficacy of other drugs evaluated in the G93A mouse model (Table 1), the magnitude of effect produced by AM-1241 and AM-630 initiated at symptom onset rivals the best yet reported for any pharmacological agent, even those given presymptomatically. AM-1241 and AM-630 produced survival interval ratios of 1.56 and 2.30, respectively, with mice living 56% longer and 130% longer, respectively, after symptom onset than controls. If extension of total life-span is considered, AM-1241 and AM-630 produced a total life-span ratio of 1.11 and 1.27, respectively (e.g., mice live 11% and 27% longer, respectively, than controls).

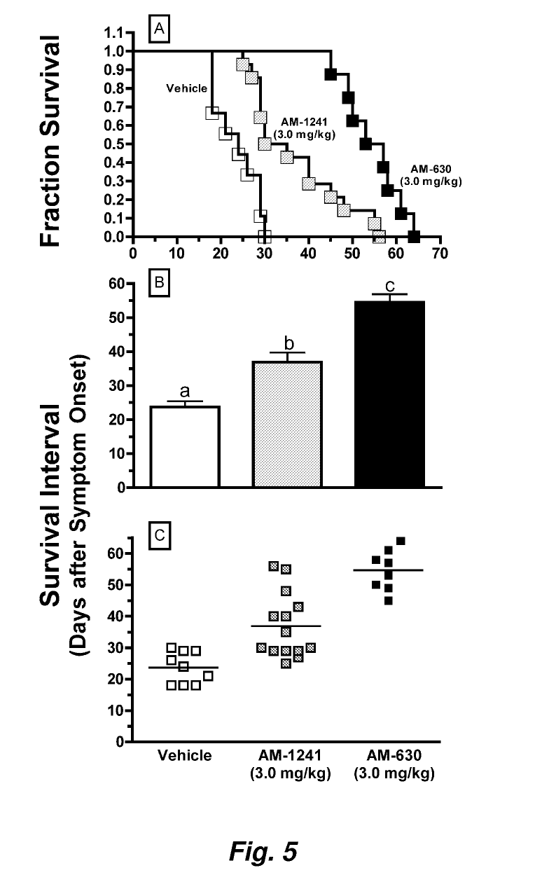

TABLE 1
Comparison of the Effect of Drugs Administered at Onset of Symptoms
on Survival Interval and Total Life-Span in G93A Mice

| | | | Survival | | |
|---|---|---|---|---|---|
| | | | Interval | | |
| | | | Ratio | Total Life-Span | |
| Drug | Route | Began | (SIR) | Ratio (TLR) | Reference |
| AM-1241 | i.p. | Onset | 1.56 | 1.11 | FIG. 4C |
| (3.0 mg/kg) | | | | | |
| AM-1241 | i.p. | Onset | 1.41 | 1.07 | FIG. 4B |
| (0.3 mg/kg) | | | | | |
| WIN-55,212-2 | i.p. | Onset | 1.37 | 1.04 | FIG. 4A |
| AM-630 | i.p. | Onset | 2.30 | 1.27 | FIG. 5A |
| (3.0 mg/kg) | | | | | |

### Example 5

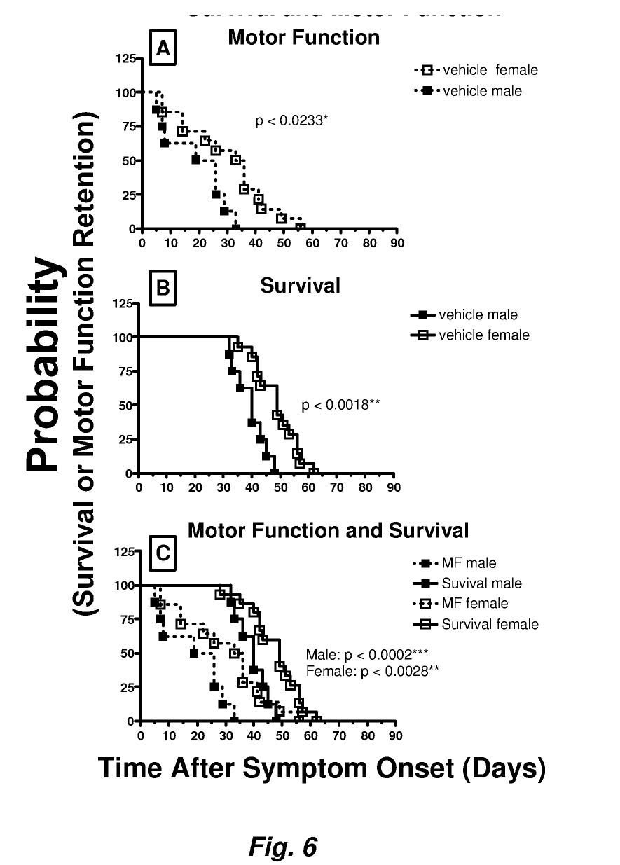

To demonstrate that CB2 antagonists would shorten the lifespan of G93A-SOD1 mice and hasten motor function decline, animals were randomly distributed into four treatment groups: vehicle, AM-1241, AM-630 and JTE-907. Testing twice a week for the duration of the study provided a quantifiable decline during disease progression. Mice were placed on a horizontal wire mesh screen that was gently inverted to 90°. Kaplan-Meier time-to-event curves were used to analyze the data. The event was defined as the day each animal was no longer able to cling to the perpendicular screen for 60 seconds. Surprisingly vehicle-treated females consistently performed better than males on the motor function test and lost their ability to cling to the screen by a mean of 29.9±4.1 days compared to 19.1±3.9 for males (p<0.0233**, FIG. 6a). The disparity between male and female motor function was paralleled by the interval between survival for the genders. Male mice survived an average of 39.6±2.0 (FIG. 6a) days past the onset of symptoms, while females lived 9 days longer than males (p<0.0018*, FIG. 6b). For males and females, loss of motor function preceded death by 20.5 (p<0.0002***) and 19.0 days (p<0.0028**), respectively.

### Example 6

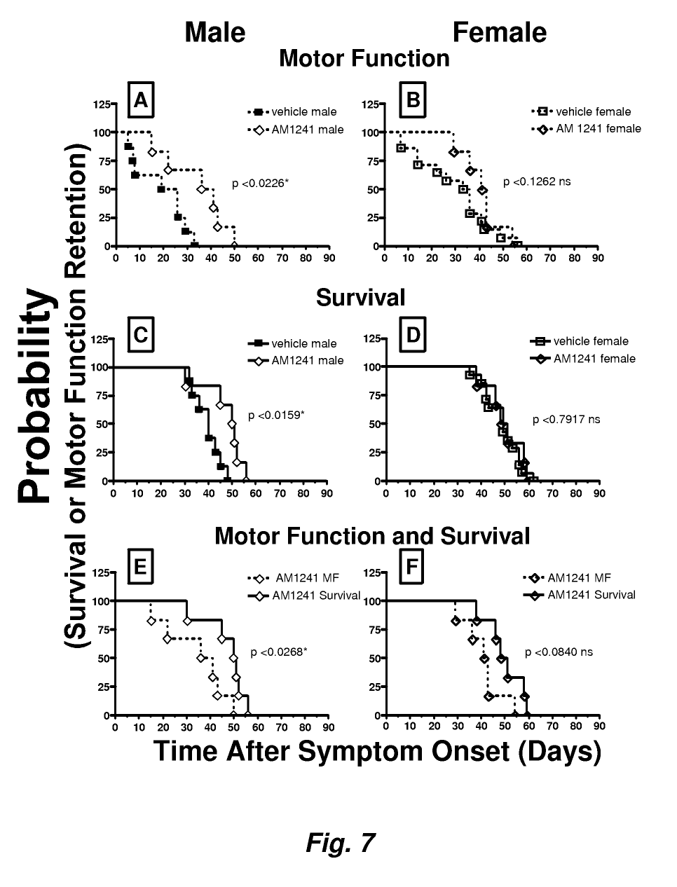

The sex-dependent difference observed for motor function and survival of vehicle-treated mice necessitated a separation of data based on gender for all conditions examined (FIGS. 6-12). Treatment with AM-1241 delayed the loss of motor function for male mice from 19.1±3.9 days to 34.5±5.5 days (FIG. 7a) after the observation of symptoms, which was an 81% improvement (p<0.0226*). AM1241-treated female mice also demonstrated a 37% improvement in motor function (FIG. 7b), although this was not statistically significant. Male mice treated with 3 mg/kg of AM-1241 lived 19% (p<0.0159*, FIG. 7c) longer, while female mice receiving the same dose did not live significantly longer than vehicle-treated mice (FIG. 7d). However, the interval between the mean loss of motor function and death was significantly decreased from 19.0 to 9.0 days for females and 20.5 to 12.8 days. This indicates that although survival was unaffected by AM-1241, both genders retain motor function longer after the onset of symptoms than vehicle-treated controls (FIGS. 7e-f).

### Example 7

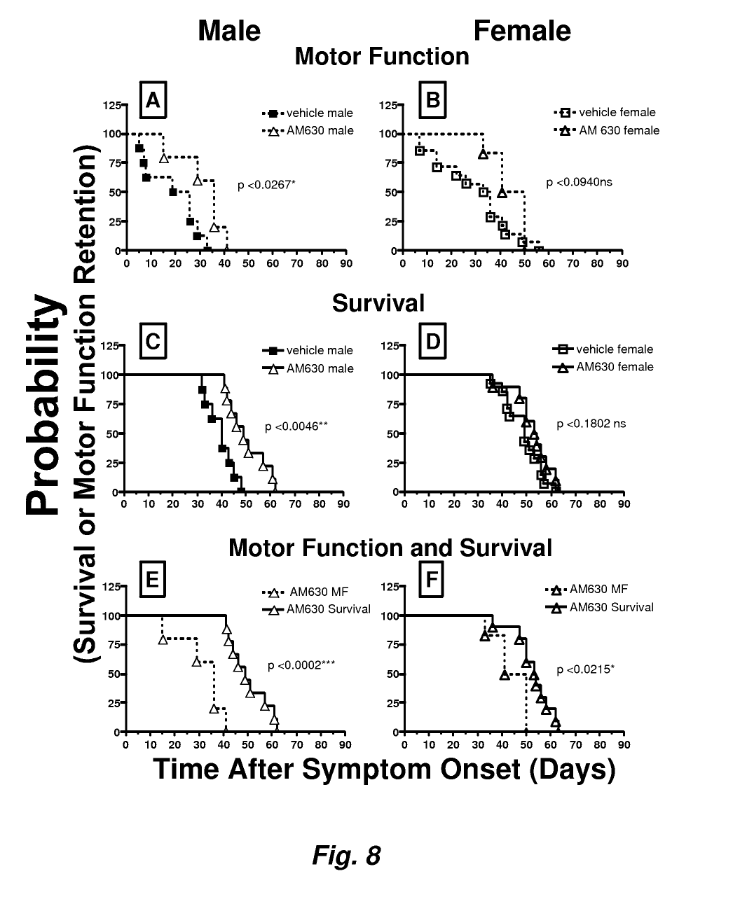

To demonstrate that activation of CB2 receptors results in prolonged survival and maintenance of motor function, a CB2 antagonist was tested which should have no benefit or shorten survival. Contrary to expectations, 3 mg/kg of daily treatment with the selective CB2 antagonist/inverse agonist AM630 treatment significantly improved survival and motor function of males by 27% (p<0.0046**, FIG. 8a) and 64% (p<0.0267*, FIG. 8c). AM630 produced a 48% improvement in motor function (48%, p<0.015*, FIG. 8b), with a non-significant 8% improvement in survival in females (FIG. 8d). The span of time between loss of gross motor function for females and death was shortened by AM630 treatment from 19.0 to 8.7 days, but for males the duration of this final period remained virtually unchanged from 20.5 to 18.9 days (FIG. 8e).

### Example 8

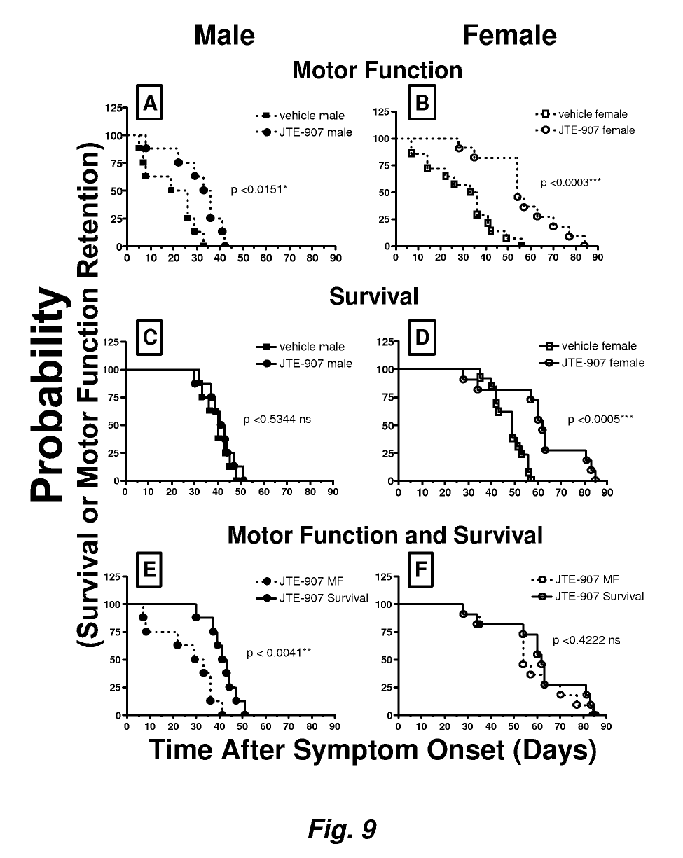

Since both AM1241 and AM630 are very similar structurally, it was considered a possibility that the beneficial effects of these ligands are not mediated via CB2 receptors. To examine this possibility, the final group was treated with JTE-907, a second selective CB2 antagonist that is structurally dissimilar to both AM1241 and AM630. Surprisingly, JTE-907 was the only putative treatment in this study that significantly improved the survival interval for females. Females lived 25% longer than age-matched vehicle treated controls (p<0.0003***, FIG. 9d). JTE-907 also maintained the motor function of female mice 92% longer than controls (p<0.0005***, FIG. 9b). Not only were survival and motor function greatly improved by JTE-907 administration, female mice also maintained the gross motor function up until just 4 days before death (FIG. 9f). Contrary to that observed in females, males treated with JTE-907 did not live significantly longer than vehicle-treated controls (FIG. 9c). However, motor function was maintained in males by JTE-907 for 61% longer (p<0.0151*, FIG. 9a). The longer maintenance of motor function observed in JTE-907 treated male mice resulted in shortening the span of time when mice could no longer cling to the wire mesh screen for 60 seconds and death from 20.5 to 10.7 days.

### Example 9

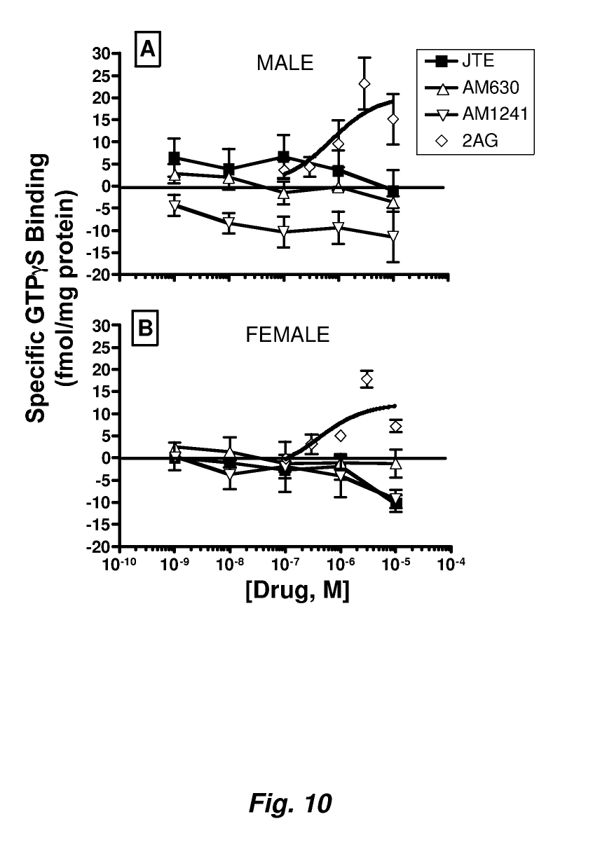

Harvested spinal cords from the vehicle treated control groups at the end-stage of their disease were separated by gender and used to conduct GTPγS binding experiments, which measure the capability of a ligand to promote or suppress G-protein activation. As an agonist, AM1241 should activate G-proteins, while AM630 and JTE-907, as antagonists/inverse agonists should either have no effect or suppress existing G-protein signaling. In direct contrast to the reported function of each ligand, all selective CB2 ligands acted as neutral antagonists in both male and female spinal cords by failing to stimulate G-proteins at concentrations ranging from 10 μM to 1 nM (FIGS. 10a-b). In males, AM1241 produced a relatively small decrease in G-protein activation, characteristic of inverse agonists. In contrast to the selective CB2 ligands, 2-AG produced a dose-related activation of G-proteins in both male and female transgenic spinal cord, producing an Eₘₐₓ of 23.21±5.82 and 17.81±1.89 with an ED₅₀ of 3.90±0.66 and 7.56±0.73, respectively. Since all selective CB2 ligands have similar intrinsic activity, these results suggest that the beneficial effects of these compounds might be due to antagonism of endogenous agonists (such as 2-AG or anandamide) rather than agonism at CB2 receptors.

### Example 10

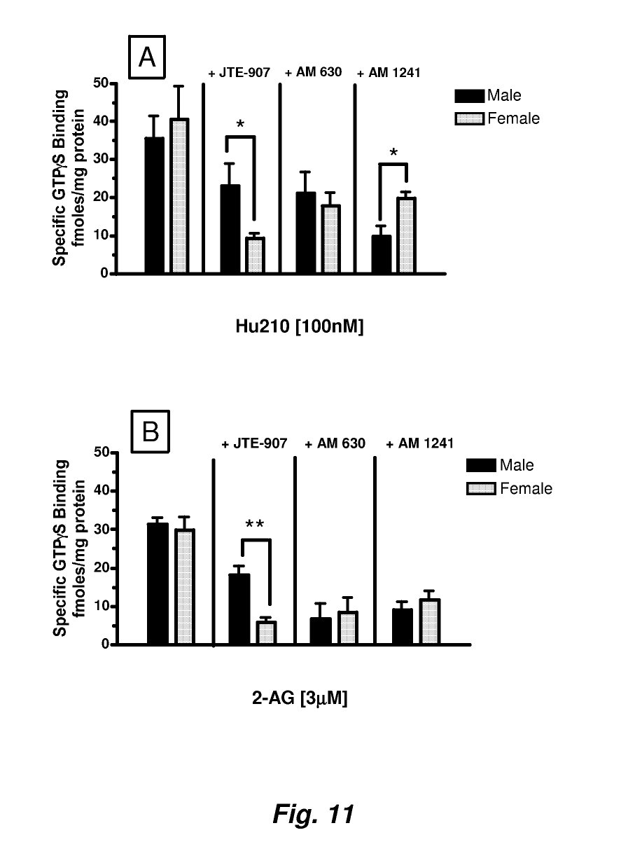

To demonstrate whether the selective CB2 ligands do indeed act as antagonists in transgenic spinal cord homogenates, each compound was evaluated for its ability to block G-protein activation by the full CB1/CB2 agonist HU-210 (FIG. 11). Stimulation of cannabinoid receptors by receptor saturating concentrations of HU-210 (100 nM) produces 35.51±5.6 and 40.59±8.5 fmole/mg protein of [³⁵S]GTP□S binding to G-proteins in male and female spinal cord homogenates, respectively (FIG. 6a, N=9). Co-incubation of 100 nM of HU-210 with receptor saturating concentrations of CB2 selective antagonist JTE-907 (1 μM) reduces G-protein stimulation to 23.3±5.6 fmole/mg protein (N=5) in male compared to 9.25±1.2 fmole/mg protein in female spinal cord homogenates (N=5, p<0.03*). Since JTE-907 treatment was markedly efficacious in female while demonstrating no effect in male mice, it is possible that JTE-907 produces greater antagonism of endocannabinoids in female relative to male mice, responsible for the beneficial effects. A receptor saturating concentration of AM630 (1 μM) reduces HU210 stimulated G-proteins in G93A male homogenates to 21.02±5.6 and to a similar level in female homogenates to 17.76±3.3 fmoles/mg protein (N=5). Finally, a receptor saturating concentration of AM1241 (1 μM) antagonizes G-protein stimulation by HU-210 to a greater degree in males (9.73±2.5 fmoles/mg protein, N=5) compared to females (19.89±1.5 fmole/mg protein, p<0.0256*, N=5). These results are in parallel with benefits observed with AM1241 treatment in males that were not seen in females.

It was next determined if the selective CB2 ligands antagonize G-protein activation produced by an endocannabinoid such as 2-AG as well. As expected, stimulation of cannabinoid receptors by the maximally efficacious concentration of 3 μM of 2-AG produces an increase in [³⁵S]GTPγS binding to G-proteins in both male and female spinal cord homogenate (31.30±1.7 and 29.93±3.3 fmole/mg protein, N=6, (FIG. 11b). Similar to that observed for HU210 a concentration of 1 μM of JTE-907 reduces 2-AG promotion of G-protein activation to 18.14±2.1 for male homogenate and 5.96±0.97 for female homogenate (p<0.0021**, N=6). AM630 (1 μM) similarly inhibits activation of G-proteins by 2-AG of homogenates prepared from both genders (males 6.66±3.9; females 8.49±3.7, N=6). Co-incubation of AM1241 (1 μM) reduces G-protein stimulation to 9.09±52.1 fmole/mg protein (N=6) for males compared to 11.62±2.2 fmole/mg protein for females (N=5, p<0.03*). These results demonstrate that there is a correlation between the degree of gender-specific inhibition by HU-210 (100 nM) or 2-AG (3 μM) stimulated G-proteins by the three CB2 selective compounds and the effectiveness of treatment.

### Example 11

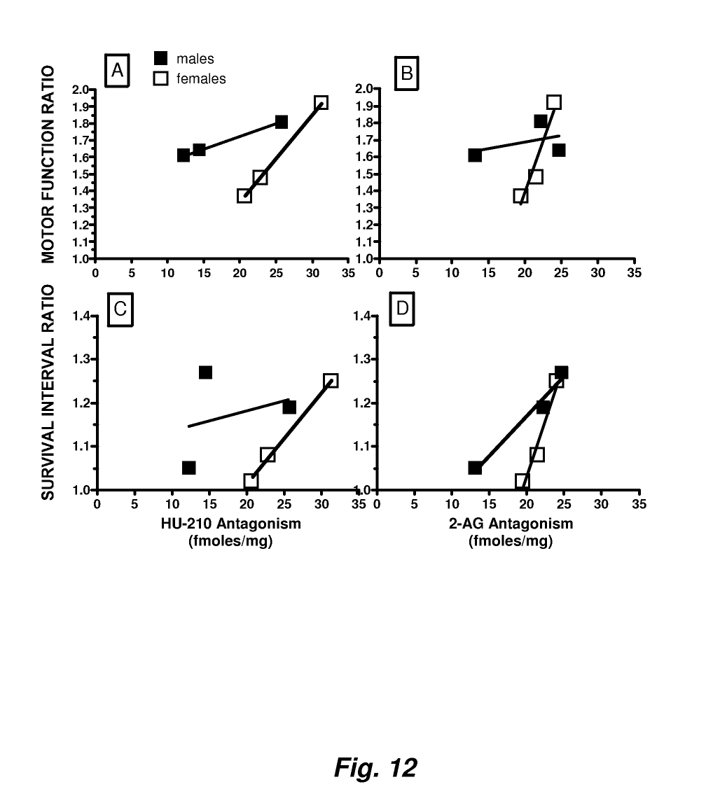

To directly compare the relationship between the therapeutic efficacy of each ligand with the degree of CB2 receptor antagonism, survival interval and motor function ratios were plotted against the amount of CB2 receptor antagonism quantified by fmole/mg protein (FIG. 12). CB2 receptor antagonism was defined by the amount of reduction in HU210 or 2-AG stimulated [³⁵S]GTPγS binding by each selective CB2 ligand. For example, JTE-907 reduces HU-210 induced G-protein activation by 12.31 fmole/mg protein in male homogenate (35.51−23.20=12.31 fmole/mg protein) and 31.34 fmole/mg protein in female homogenate (40.59−9.25=27.9 fmole/mg protein). In FIG. 12a the r² value for the relationship HU210 antagonism and improvement in motor function is 0.9999 for males and 1.000 for females. The correlation between HU210 antagonism and survival interval ratio was 0.9959 for females and 0.091 for males, although this low value could be due to one data point skewing the curve (FIG. 12c). The correlation between antagonism 2-AG and motor function and survival is 0.1971 and 0.9732 for males and 0.9286 and 0.9580 for females (FIG. 12b). In conclusion, FIG. 12 depicts a direct correlation between the antagonism by these three compounds of either and endogenous or exogenous agonists and increases of motor function and survival interval ratios.

All references cited in this specification are hereby incorporated by reference in their entirety. The discussion of the references herein is intended merely to summarize the assertions made by their authors and no admission is made that any reference constitutes prior art relevant to patentability. Applicant reserves the right to challenge the accuracy and pertinence of the cited references.

As various changes could be made in the above methods and compositions without departing from the scope of the invention, it is intended that all matter contained in the above description be interpreted as illustrative and not in a limiting sense. Unless explicitly stated to recite activities that have been done (i.e., using the past tense), illustrations and examples are not intended to be a representation that given embodiments of this invention have, or have not, been performed.

### REFERENCES

Bilsland L. G., Dick J. R., Pryce G., Petrosino S., Di Marzo V., Baker D. and Greensmith L. (2006) Increasing cannabinoid levels by pharmacological and genetic manipulation delay disease progression in SOD1 mice. FASEB J.
Breivogel C. S., Griffin G., Di Marzo V. and Martin B. R. (2001) Evidence for a new G protein-coupled cannabinoid receptor in mouse brain. Mol. Pharmacol. 60, 155-163.
Felder C. C., Joyce K. E., Briley E. M., Mansouri J., Mackie K., Blond O., Lai Y., Ma A. L. and Mitchell R. L. (1995) Comparison of the pharmacology and signal transduction of the human cannabinoid CB1 and CB2 receptors. Mol. Pharmacol. 48, 443-450.
Gardner A. and Mallet P. E. (2006) Suppression of feeding, drinking, and locomotion by a putative cannabinoid receptor ‘silent antagonist’. Eur. J. Pharmacol. 530, 103-106.
Gatley S. J., Lan R., Pyatt B., Gifford A. N., Volkow N. D. and Makriyannis A. (1997) Binding of the non-classical cannabinoid CP 55,940, and the diarylpyrazole AM251 to rodent brain cannabinoid receptors. Life Sci. 61, PL 191-197.
Giulietti A., Overbergh L., Valckx D., Decallonne B., Bouillon R. and Mathieu C. (2001) An overview of real-time quantitative PCR: applications to quantify cytokine gene expression. Methods 25, 386-401.
Gurney M. E. (1997) Transgenic animal models of familial amyotrophic lateral sclerosis. J. Neurol. 244 Suppl 2, S15-20.
Hanisch U. K. (2002) Microglia as a source and target of cytokines. Glia 40, 140-155.
Hillard C. J., Manna S., Greenberg M. J., DiCamelli R., Ross R. A., Stevenson L. A., Murphy V., Pertwee R. G. and Campbell W. B. (1999) Synthesis and characterization of potent and selective agonists of the neuronal cannabinoid receptor (CB1). J. Pharmacol. Exp. Ther. 289, 1427-1433.
Hosohata K., Quock R. M., Hosohata Y., Burkey T. H., Makriyannis A., Consroe P., Roeske W. R. and Yamamura H. I. (1997) AM630 is a competitive cannabinoid receptor antagonist in the guinea pig brain. Life Sci. 61, PL115-118.
Ince P. G., Shaw P. J., Slade J. Y., Jones C. and Hudgson P. (1996) Familial amyotrophic lateral sclerosis with a mutation in exon 4 of the Cu/Zn superoxide dismutase gene: pathological and immunocytochemical changes. Acta Neuropathol. (Berl.) 92, 395-403.
Iwamura H, Suzuki H, Ueda Y, Kaya T, Inaba T (2001) In vitro and in vivo pharmacological characterization of JTE-907, a novel selective ligand for cannabinoid CB2 receptor. J Pharmacol Exp Ther 296: 420-425
Nelson P. T., Soma L. A. and Lavi E. (2002) Microglia in diseases of the central nervous system. Ann. Med. 34, 491-500.
Prather P. L., Martin N. A., Breivogel C. S, and Childers S. R. (2000a) Activation of cannabinoid receptors in rat brain by WIN 55212-2 produces coupling to multiple G protein alpha-subunits with different potencies. Mol. Pharmacol. 57, 1000-1010.
Prather P. L., Song L., Piros E. T., Law P. Y. and Hales T. G. (2000b) δ-Opioid receptors are more efficiently coupled to adenylyl cyclase than to L-type Ca²⁺ channels in transfected rat pituitary cells. J. Pharmacol. Exp. Ther. 295, 552-562.
Raman C., McAllister S. D., Rizvi G., Patel S. G., Moore D. H. and Abood M. E. (2004) Amyotrophic lateral sclerosis: delayed disease progression in mice by treatment with a cannabinoid. Amyotroph. Lateral Scler. Other Motor Neuron Disord. 5, 33-39.
Rinaldi-Carmona M., Barth F., Millan J., Derocq J. M., Casellas P., Congy C., Oustric D., Sarran M., Bouaboula M., Calandra B., Portier M., Shire D., Breliere J. C. and Le Fur G. L. (1998) SR 144528, the first potent and selective antagonist of the CB2 cannabinoid receptor. J. Pharmacol. Exp. Ther. 284, 644-650.
Shoemaker J. L., Joseph B. K., Ruckle M. B., Mayeux P. R. and Prather P. L. (2005) The endocannabinoid noladin ether acts as a full agonist at human CB2 cannabinoid receptors. J. Pharmacol. Exp. Ther. 314, 868-875.
Streit W. J. (2002) Microglia as neuroprotective, immunocompetent cells of the CNS. Glia 40, 133-139.
Strong M. and Rosenfeld J. (2003) Amyotrophic lateral sclerosis: a review of current concepts. Amyotroph. Lateral Scler. Other Motor Neuron Disord. 4, 136-143.
Valenzano K. J., Tafesse L., Lee G., Harrison J. E., Boulet J. M., Gottshall S. L., Mark L., Pearson M. S., Miller W., Shan S., Rabadi L., Rotshteyn Y., Chaffer S. M., Turchin P. I., Elsemore D. A., Toth M., Koetzner L. and Whiteside G. T. (2005) Pharmacological and pharmacokinetic characterization of the cannabinoid receptor 2 agonist, GW405833, utilizing rodent models of acute and chronic pain, anxiety, ataxia and catalepsy. Neuropharmacol. 48, 658-672.
Weydt P., Hong S., Witting A., Moller T., Stella N. and Kliot M. (2005) Cannabinol delays symptom onset in SOD1 (G93A) transgenic mice without affecting survival. Amyotroph. Lateral Scler. Other Motor Neuron Disord. 6, 182-184.
Witting A., Weydt P., Hong S., Kliot M., Moller T. and Stella N. (2004) Endocannabinoids accumulate in spinal cord of SOD1 G93A transgenic mice. J. Neurochem. 89, 1555-1557.
Yao B. B., Mukherjee S., Fan Y., Garrison T. R., Daza A. V., Grayson G. K., Hooker B. A., Dart M. J., Sullivan J. P. and Meyer M. D. (2006) In vitro pharmacological characterization of AM1241: a protean agonist at the cannabinoid CB(2) receptor? Br. J. Pharmacol.

What is claimed is:

1-8. (canceled)

9. A method of treating a neurodegenerative disease in a mammal comprising administering a selective CB2 receptor modulator to a patient in need thereof.

10. The method of claim 9 wherein said neurodegenerative disease is selected from the group consisting of amyotrophic lateral sclerosis, Alzheimer's disease, Parkinson's disease, Huntington's disease and multiple sclerosis.

11. The method of claim 9 wherein said neurodegenerative disease is amyotrophic lateral sclerosis.

12. The method of claim 9 wherein said selective CB2 receptor modulator is selected from the group consisting of a selective CB2 receptor partial agonist, a selective CB2 receptor antagonist and a selective CB2 receptor inverse agonist.

13. The method of claim 12 wherein said selective CB2 receptor modulator is a selective CB2 receptor partial agonist.

14. The method of claim 12 wherein said selective CB2 receptor modulator is a selective CB2 receptor antagonist.

15. The method of claim 12 wherein said selective CB2 receptor modulator is a selective CB2 receptor inverse agonist.

16. The method of claim 9 wherein said CB2 receptor modulator is administered in a dose and wherein said dose is in the range of 0.3 mg/kg/day to 5.0 mg/kg/day.

17. The method of claim 16 wherein administration of the dose is via a method selected from the group consisting of oral, parenteral, intravenous, intramuscular, topical and subcutaneous

18-25. (canceled)

26. The method of claim 9 wherein the selective CB2 receptor modulator is selected from the group consisting of AM-1241; GW-405833; JWH-015, WIN-55,212-3; AM-630; SR-144528; and JTE-907.


SEQUENCE LISTING (6 sequences)

SEQ ID NO: 1 - DNA, 19 bp, Homo sapiens
ctgaactccaccgtgaacc

SEQ ID NO: 2 - DNA, 18 bp, Homo sapiens
ttattggcgtgcttgtgc

SEQ ID NO: 3 - DNA, 17 bp, Homo sapiens
tgcactggctctcatgg

SEQ ID NO: 4 - DNA, 19 bp, Homo sapiens
gagcgaatctctccactcc

SEQ ID NO: 5 - DNA, 19 bp, Homo sapiens
gggaagctcactggcatgg

SEQ ID NO: 6 - DNA, 26 bp, Homo sapiens
cttcttgatgtcatcatacttggcag